(19) United States
(12) Patent Application Publication
(10) Pub. No.: US 2024/0002348 A1
(43) Pub. Date: Jan. 4, 2024

(54) QUINOLONE COMPOUNDS AND PROCESS FOR PREPARATION THEREOF

(71) Applicant: COUNCIL OF SCIENTIFIC AND INDUSTRIAL RESEARCH (AN INDIAN REGISTERED BODY INCORPORATED UNDER THE REGN, New Delhi, Delhi (IN)

(72) Inventors: Srihari PABBARAJA, Telangana Hyderabad (IN); Goverdhan MEHTA, Telangana Hyderabad (IN); Shweta SINGH, Telangana Hyderabad (IN)

(21) Appl. No.: 18/042,570

(22) PCT Filed: Aug. 17, 2021
(86) PCT No.: PCT/IN2021/050790

§ 371 Date: Feb. 22, 2023

(30) Foreign Application Priority Data
Aug. 22, 2020 (IN) 202011036288

(51) Int. Cl. C07D 215/233; C07D 471/04; C07D 491/056; C07D 405/08

(52) U.S. Cl. CPC C07D 215/233; C07D 405/08; C07D 491/056; C07D 471/04

(57) ABSTRACT

The present invention relates to quinolones of formula (I) and process for its preparation by amine insertion into aryl-ynones thereof. [Formula I] The invention further relates to the process to obtain the natural products such as: graveoline, graveolinine, pseudane IV, pseudane VII, pseudane VIII and pseudane XII. The invention also describes the process for the total synthesis of waltherione F in concise approach from the quinolone synthesized. [Formula II]

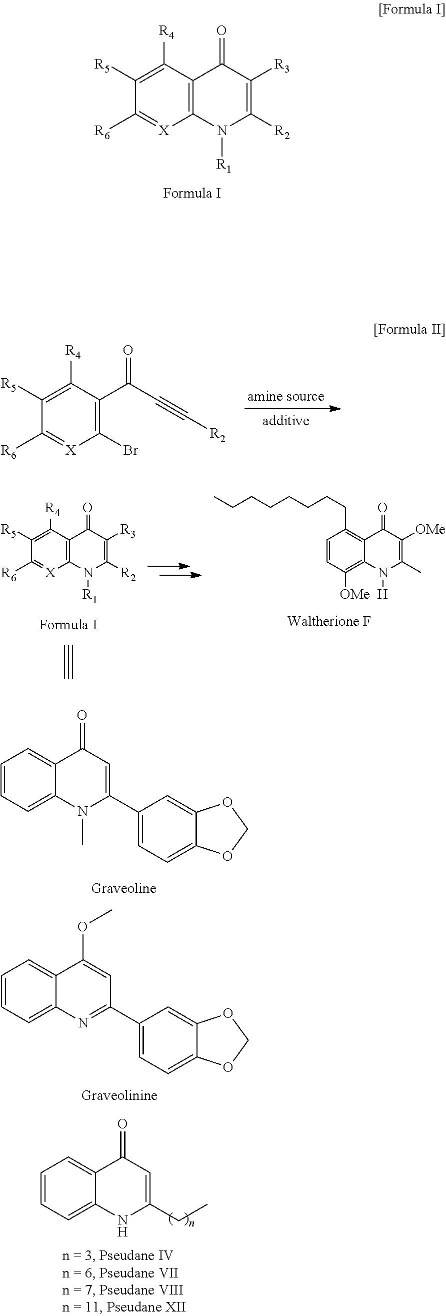

### FIELD OF THE INVENTION

The present invention relates to quinolones and analogs of Formula (I); and process for it's preparation by amine insertion into aryl-ynones thereof

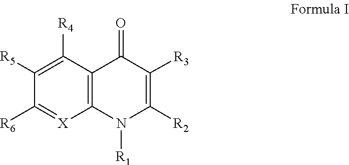

wherein X═C, N, C—OMe, R₁═H, CH₃, R₂═C₁-C₁₂ alkyl, cyclopropyl, cyclohexyl, phenyl, 2-fluoro phenyl, 4-fluoro phenyl, 4-methoxy phenyl, 4-ethyl phenyl, 3,4-methylenedioxyphenyl. R₃═H, OMe, R₄═H, C₁-C₈ alkyl, Bromo, R₅═H, R₆═H, R₅ and R₆ can be taken together to form —OCH₂O—.

The invention also relates to the process for the preparation of graveoline (1), graveolinine (2), pseudane IV (3), pseudane VII (4), pseudane VIII (5), pseudane XII (6) and waltherione F (7) as quinolones of general formula (I).

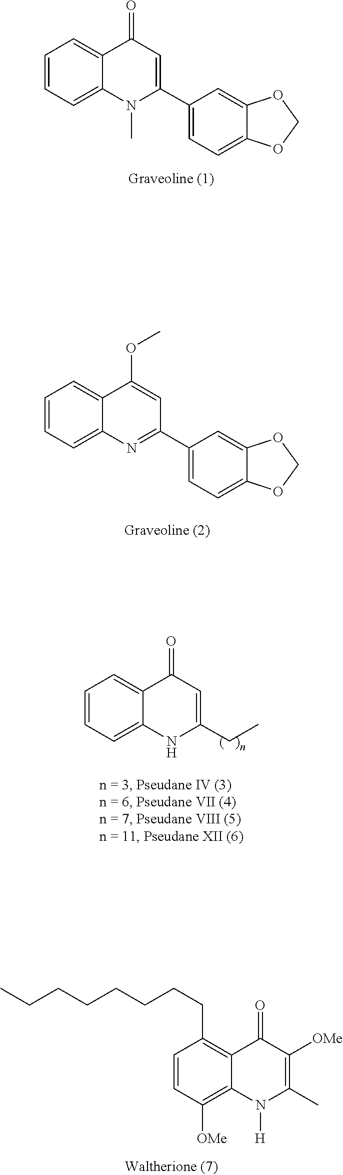

In particular, the invention also relates to the total synthesis of graveoline (1), graveolinine (2), pseudane IV (3), pseudane VII (4), pseudane VIII (5), pseudane XII (6) and waltherione F(7) of general formula I.

### BACKGROUND OF THE INVENTION

The present protocol focuses on the synthesis of bicyclic nitrogen containing heterocyclic compounds called quinolones. Quinolone and its derivatives have attracted significant attention due to their widespread occurrence in several natural products, pharmaceuticals and exhibition of wide profile of biological properties (J. Med. Chem. 2014, 57, 1952, Chem. Rev. 2011, 111, 152). The 4-quinolone ring is a common motif present in several alkaloids and serves as an important motif in drugs that show important pharmaceutical activities and hence considered as privileged building block for pharmaceutics. Importance of quinolone and its derivatives as different therapeutic agents is well precedented in as anti-tumor agents (US2005/0032832, WO 96/10563), antimitotic (WO02/26730, Eur. J. Med. Chem. 2011, 46, 6046), antimalarial (J. Med. Chem. 2014, 57, 3818), antiviral agents, xanthine oxidase and cathepsins inhibitory activities (Arch. Pharm. 2013, 346, 7), auto inducers (WO 02/18342), inhibitors for formation of C-MYC/MAX/DNA complex (WO 2018/021810 A1), anti-bacterial or anti-fungal agents, zinc sensors (WO 2017/017631A2, WO 2017/220205 A1), lysyl oxidase-like 2 (LOXL2) inhibitors (WO 2017/139274 A1), treating apicomplexan parasite related disorders (WO2017/112678 A1), inhibitors of activity of tyrosinase and related proteins (WO 2017/181379 A1), work as allosteric modulators for treatment of diseases such as Alzheimer's disease, schizophrenia pain or sleep disorders (WO 2017/160670 A1). Autoinducer which act as intercellular signal molecule in the cell to cell communication system of Pseudomonas aeruginosa (WO 02/18342 A2) etc.

The classic methods for the synthesis of 4-quinolinones include Lappin cyclization (J. Am. Chem. Soc. 1948, 70, 3348), Niementowski method (Tetrahedron Lett. 2002, 43, 3911), Conrad-Limpet method (Eur. J. Org. Chem. 2010, 2010, 5841), Camps cyclization (Chem. Ber. 1899, 32, 3228, Org. Lett. 2008, 10, 2609) and Grohe-Heitzer synthesis (Liebigs Ann. Chem. 1987, 1987, 29). In many of the synthesis, multistep procedure is involved to build enaminone precursor and also high temperature is required for the cyclization to occur. The Camps approach is the condensation of aniline with Meldrum's acid (or its derivatives) and trimethyl orthoformate to afford the corresponding enamine which is then cyclized in high boiling solvents (Synthesis 1987, 482) or under microwave conditions (Bioorg. Med. Chem. Lett. 2005, 15, 1015) to achieve the cyclization and yield the quinolones. Apart from the above, there are several other reports for the synthesis of quinolones using transition metal catalysts (J. Org. Chem. 2007, 72, 7968, Eur. J. Org. Chem. 2012, 3001, Eur. J. Org. Chem. 2014, 4044). In synthesis, few procedures involve high pressures or toxic carbon monoxide (Chem. Heterocycl. Compd. 2009, 45, 757) or sometimes, the scarcely available N-(o-ketoaryl)amides which forms the limiting factors/bottle necks for the synthesis of quinolones. Owing to the importance of quinolones and their derivatives, several groups became interested by focusing on one-pot procedures for the synthesis of quinolones. Few multi-component methods towards accessing the quinolones include copper catalysed three component synthesis with substituted 3-(2-halophenyl)-3-oxopropane, aldehydes and aq. NH₃ using water as solvent media (Adv. Synth. Catal. 2019, 361, 1-15), aminoacylation of ynones with amides to get substituted-3-aroylquinolin-4(1H)-one scaffolds (Org. Lett. 2010, 12, 212, J. Org. Chem. 2016, 81, 12181, Org. Lett. 2018, 20, 3907), ortho-functionalization of anilines with alkynes or alkenes and Aza-Michael addition alternative approaches (J. Org. Chem. 2015, 80, 1464, J. Org. Chem. 2018, 83, 2694). Carbonylative coupling of o-iodoaniline with terminal acetylene and carbon monoxide in presence of palladium catalyst (Tetrahedron Lett. 1991, 32, 237) etc. In view of the wide variety of applications for quinolones, the development of new processes overcoming the general challenges and eco-friendly strategy for their accessibility in focusing on one-pot procedures is highly desirable.

### Objective of the Invention

Main objective of the present invention is to provide novel quinolones and analogs of formula (I).

Another objective of the present invention is to provide an efficient process for the preparation of quinolones and analogs of formula (I), by amine insertion method.

Another objective of the present invention is to provide a process, which could be carried out by employing a protocol using additive and ammonia source in one-pot approach using pre-installed ynones of formula (II).

Another objective of the present invention is the total synthesis of natural products, for example: graveoline (1), graveolinine (2), pseudane IV (3), pseudane VII (4), pseudane VIII (5), and pseudane XII (6).

Another objective of the present invention is to extend the strategy and utilize one of the obtained quinolone products for the total synthesis of natural product waltherione F (7) in a concise approach.

### SUMMARY OF THE INVENTION

Accordingly, the present invention provides compound of formula (I):

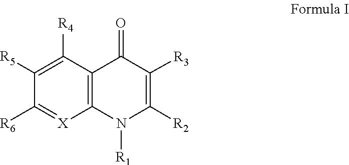

wherein;
X is C, N, and C—OMe,
R₁ is H, and CH₃,
R₂ is C₁-C₁₂ alkyl, cyclopropyl, cyclohexyl, phenyl, 2-fluoro phenyl, 4-fluoro phenyl, 4-methoxy phenyl, 4-ethyl phenyl, and 3,4-methylenedioxyphenyl,
R₃ is H, Br, and OMe,
R₄ is H, C₁-C₈ alkyl, and bromo,
R₅ and R₆ is H, and
R₅ and R₆ can be taken together to form —OCH₂O—.

In an embodiment, the present invention provides a process for the preparation of quinolones of general formula (I) by amine insertion method, comprising the steps as described in the detailed description.

In a preferred embodiment the present invention provides a compound of formula (2g)

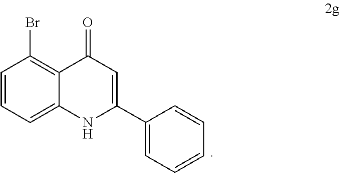 [SMILES: O=c1cc(-c2ccccc2)[nH]c2cccc(Br)c12]

In an embodiment, the present invention provides compound of general formula (II):

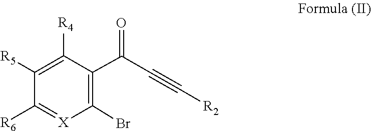

wherein, substituents R₂, R₄, R₅, R₆ and X are same as defined above.

In another embodiment, the present invention provides, process for the preparation of quinolones of general formula (I) comprising; treatment of ynones of formula (II) with ammonia source such as ammonium carbonate, ammonia in presence of metal halide as an additive in polar solvent such as DMF or formamide at about 80-120° C. for about 8-15 h.

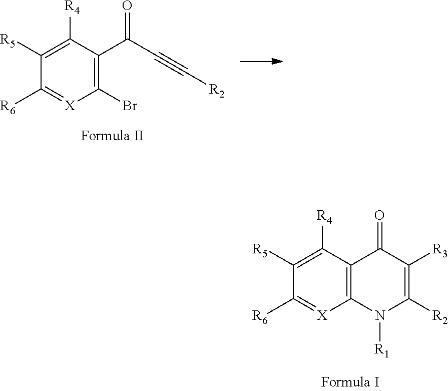

In another embodiment, the present invention provides a process for the preparation of graveoline (1), graveolinine (2), pseudane IV (3), pseudane VII (4), pseudane VIII (5), and pseudane XII (6) having following formulae.

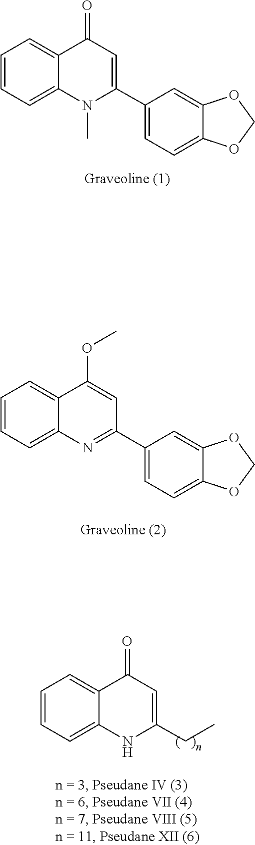

In another embodiment, the present invention provides a process for the preparation of waltherione F of formula (7) involving quinolone of general formula (I) as an intermediate.

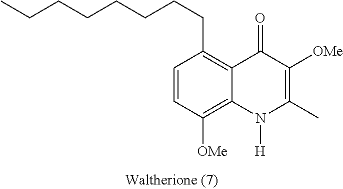

In yet another embodiment, the present invention provides, process for the preparation of waltherione F of formula (7) comprising the following steps.

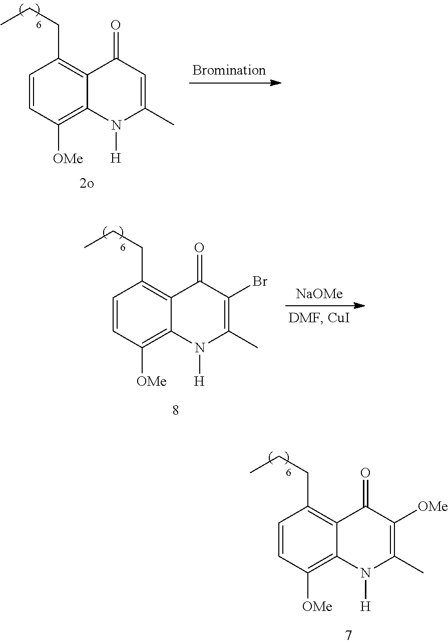

### DETAILED DESCRIPTION OF THE INVENTION

The present invention provides a new and efficient processes, and intermediates thereof for the preparation of quinolones and its derivatives

The strategy of present invention is extended to utilize one of the obtained quinolone product for the total synthesis of natural product, such as, but not limited to: graveoline (1), graveolinine (2), pseudane IV (3), pseudane VII (4), pseudane VIII (5), pseudane XII (6) and waltherione F (7).

As used herein, the modifier “about” should be considered as disclosing the range defined by the absolute values of the two endpoints. For example, the expression “from about 1 to about 4” also discloses the range “from 1 to 4.” When used to modify a single number, the term “about” may refer to ±10% of the said number including the indicated number. For example, “about 10%” may cover a range of 9% to 11%, and “about 1” means from 0.9-1.1.

In an embodiment, the present invention provides compound of following formula (I):

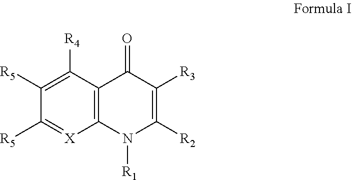

wherein;
X is C, N, and C—OMe,
R₁ is H, and CH₃,
R₂ is C₁-C₁₂ alkyl, cyclopropyl, cyclohexyl, phenyl, 2-fluoro phenyl, 4-fluoro phenyl, 4-methoxy
phenyl, 4-ethyl phenyl, and 3,4-methylenedioxyphenyl,
R₃ is H, Br, and OMe,
R₄ is H, C₁-C₈ alkyl, and bromo,
R₅ and R₆ is H, and
R₅ and R₆ can be taken together to form is —OCH₂O—.

In another embodiment, the compound of formula (I) is selected from:

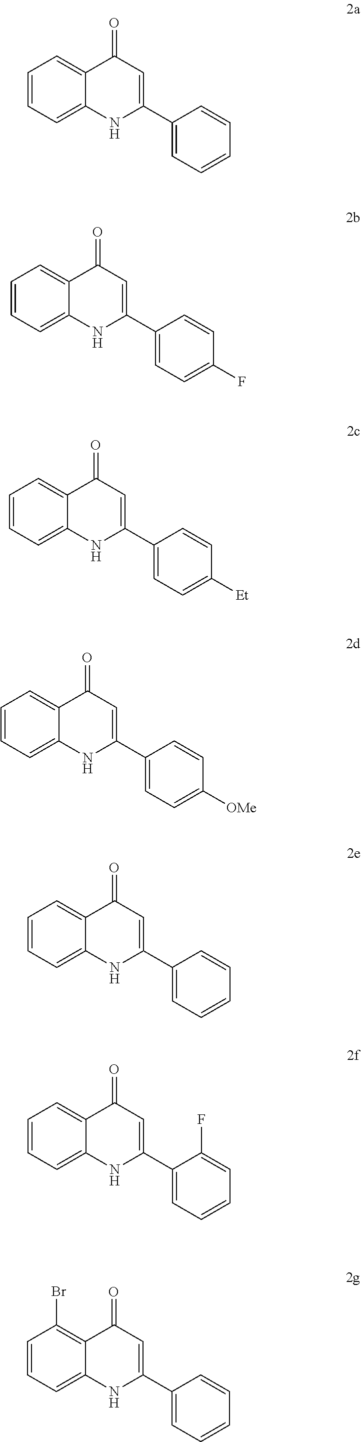

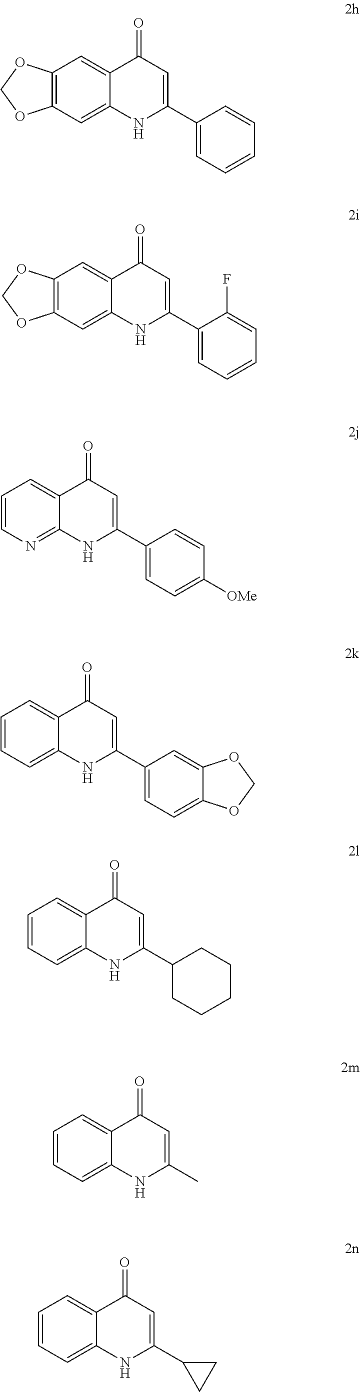

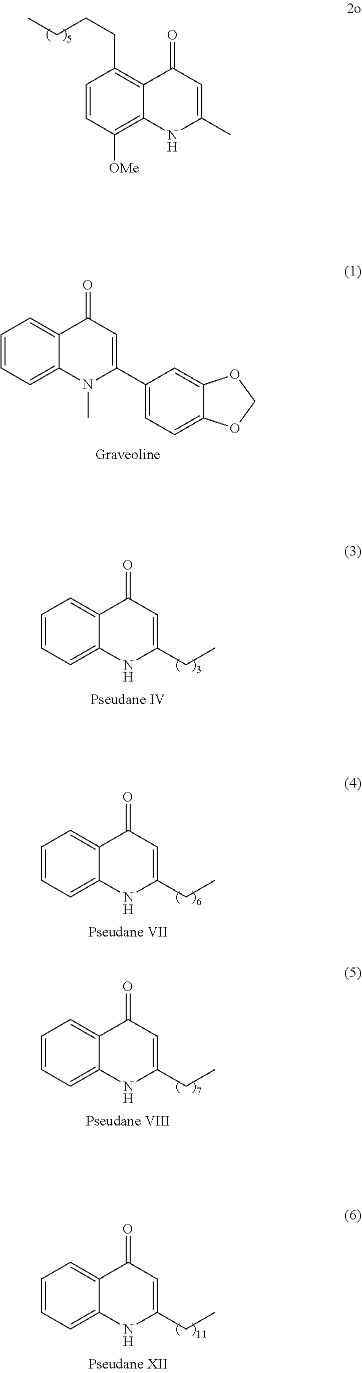

In most preferred embodiment the compound of formula (I) is:

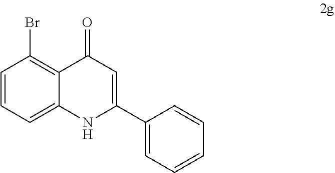 [SMILES: O=c1cc(-c2ccccc2)[nH]c2cccc(Br)c12]

In an embodiment, the present invention provides a compound Graveolinine (2) derived from compound 2k of formula (I)

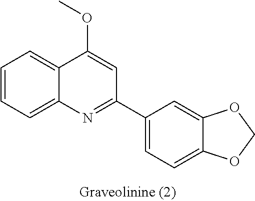 [SMILES: COc1cc(-c2ccc3c(c2)OCO3)nc2ccccc12]

In an embodiment, the present invention provides process for the preparation of quinolones of general formula (I) by amine insertion method.

The present process could be operated by employing the protocol using additive and ammonia source in one-pot approach using pre-installed ynones in high yields and purity. This newly developed process starts from a pre-installed ynone (formula I) as illustrated in scheme 1.

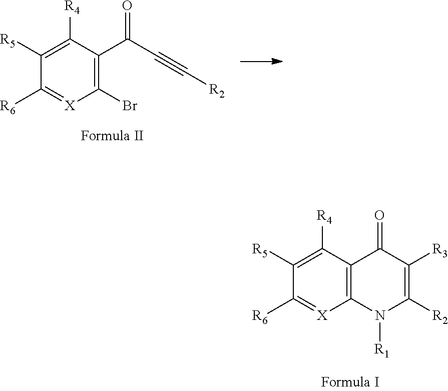

Scheme 1: Synthesis of formula I from formula II
wherein;
X is C, N, and C—OMe,
R₁ is H, and CH₃,
R₂ is C₁-C₁₂ alkyl, cyclopropyl, cyclohexyl, phenyl, 2-fluoro phenyl, 4-fluoro phenyl, 4-methoxy phenyl, 4-ethyl phenyl, and 3,4-methylenedioxyphenyl,
R₃ is H, Br, and OMe,
R₄ is H, C₁-C₈ alkyl, and bromo,
R₅ and R₆ is H, and
R₅-R₆ is —OCH₂O—.

The present process can be performed very effectively in with a wide range of substrates and is a highly viable strategy which could be most suitable for the industrial scale production of quinolones and analog. Further, this process is most suitable for the generation of a large library of intermediates and related molecules containing quinolone moieties. All the reactions/experiments involve purification and systematic characterization of the individual reaction product as represented in the general process.

The present process for the preparation of quinolones and analogs, in particular quinolones and analogs by amine insertion method as illustrated in scheme 1 is the most convenient and simple method involving a protocol using additive and ammonia source and other reaction parameters.

Particularly, the reaction of compound of formula (II) bromoaryl ynones with inorganic base such as K₂CO₃ or Cs₂CO₃ or Na₂CO₃ in polar solvents such as DMF or formamide or DMSO or Dioxane and heating the mixture along with ammonium acetate or ammonium carbonate in presence of copper(I) iodide provides the quinolones of formula (I).

In a preferred embodiment, the present invention provides process for the preparation of quinolones of general formula (I) comprising; treatment of ynones of formula (II) with ammonia source such as ammonium carbonate, ammonia in presence of metal halide as an additive in polar solvent such as DMF or formamide at about 80-120° C. for about 8-15 h.

In an embodiment, the present invention provides a process for the preparation of quinolones of general formula (I), wherein the metal halide as an additive is selected from copper iodide, copper bromide, and copper chloride.

In an embodiment, the present invention provides compound of general formula (II):

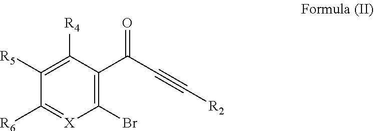

wherein, substituents R₂, R₄, R₅, R₆ and X are same as defined above.

In another embodiment, the compound of formula (II) is selected from:

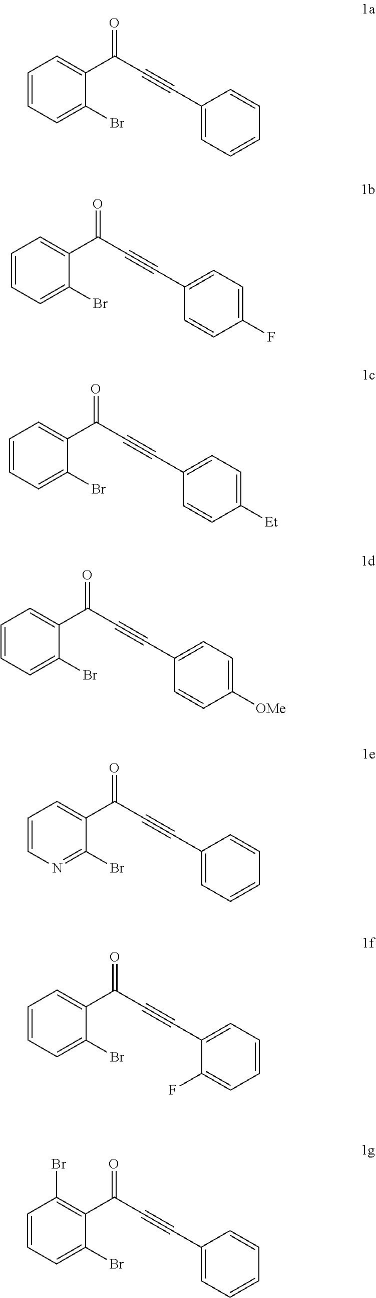

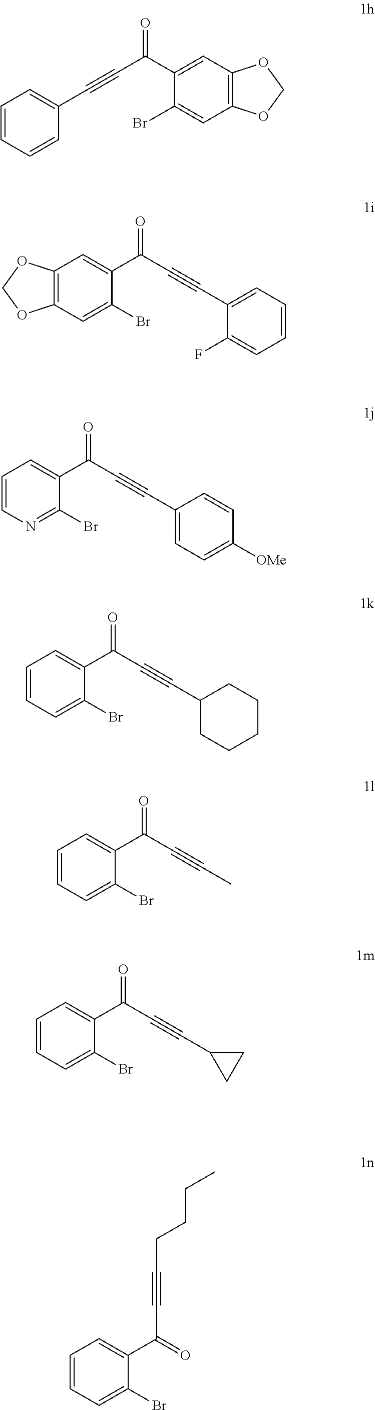

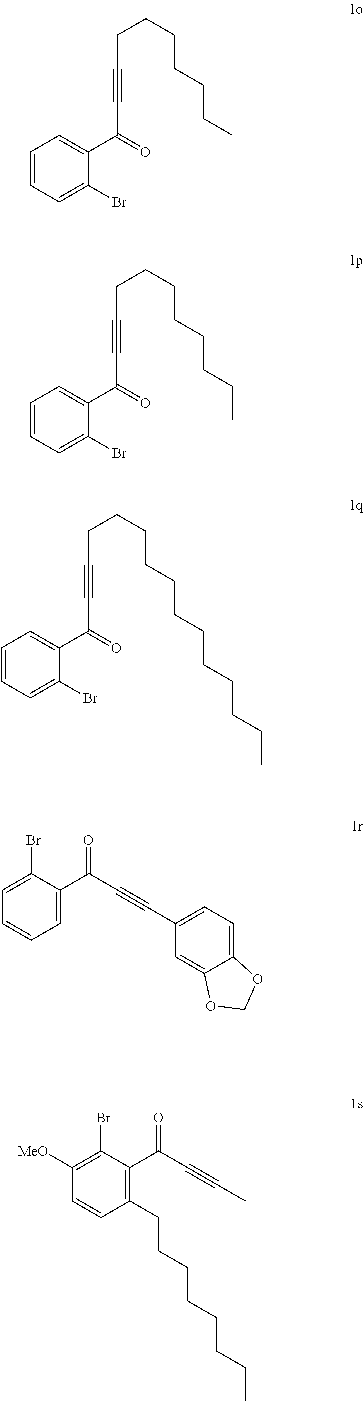 [SMILES: CCCCCCCC#CC(=O)c1ccccc1Br ; CCCCCCCCC#CC(=O)c1ccccc1Br ; CCCCCCCCCCCCC#CC(=O)c1ccccc1Br ; O=C(C#Cc1ccc2c(c1)OCO2)c1ccccc1Br ; CC#CC(=O)c1c(CCCCCCCC)ccc(OC)c1Br]

In most preferred embodiment the compounds of formula (II) are:

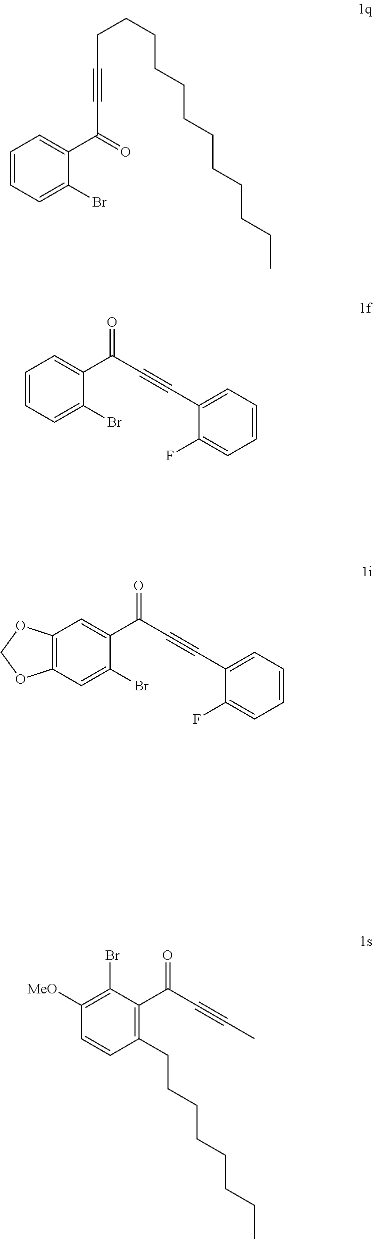 [SMILES: CCCCCCCCCCCCC#CC(=O)c1ccccc1Br ; O=C(C#Cc1ccccc1F)c1ccccc1Br ; O=C(C#Cc1ccccc1F)c1cc2c(cc1Br)OCO2 ; CC#CC(=O)c1c(CCCCCCCC)ccc(OC)c1Br]

In another embodiment, the present invention provides a process for the preparation of graveoline (1), graveolinine (2), pseudane IV (3), pseudane VII (4), pseudane VIII (5), and pseudane XII (6) having general formula (I).

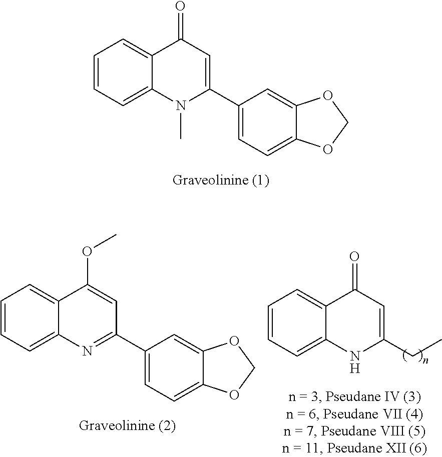

In another embodiment, the present invention provides process for the preparation of graveoline (1), graveolinine (2), pseudane IV (3), pseudane VII (4), pseudane VIII (5) and pseudane XII (6) of general formula (I) comprising of treatment of ynones of formula (II) with ammonia source such as ammonium carbonate, ammonia in presence of metal halide as an additive in polar solvent such as DMF or formamide.

In an embodiment, the present invention provides a process for the preparation of graveoline (1), graveolinine (2), pseudane IV (3), pseudane VII (4), pseudane VIII (5) and pseudane XII (6) of general formula (I), wherein the metal halide as an additive is selected from copper iodide, copper bromide, and copper chloride.

In another embodiment, the present invention provides process for the preparation of waltherione F of formula (7) involving quinolone of general formula (I) as an intermediate.

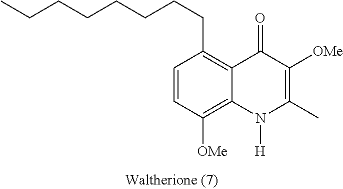

In yet another embodiment, the present invention provides, process for the preparation of waltherione F of formula (7) comprising the following steps.

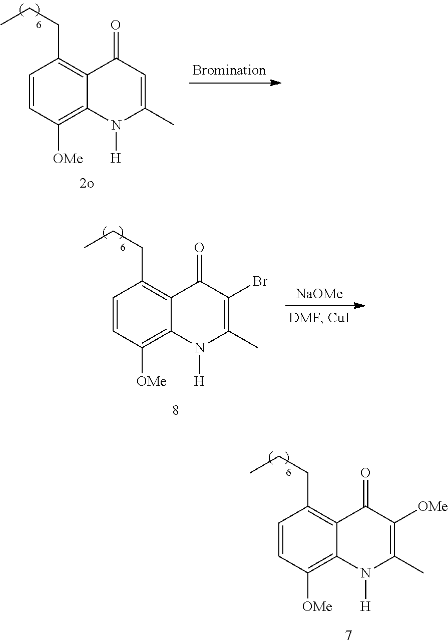

In another embodiment, the present invention provides, process for the preparation of 8-methoxy-2-methyl-5-octylquinolin-4(1H)-one (2o), in particular; and its utility as an intermediate for the total synthesis of waltherione F (7).

In another embodiment, the present invention provides a process for the preparation of waltherione F (7) comprising of the steps; subjecting 8-methoxy-2-methyl-5-octylquinolin-4(1H)-one (2o) to bromination to provide the corresponding brominated product (8), and treating brominated product (8) with sodium methoxide in presence of copper iodide to provide waltherione F(7).

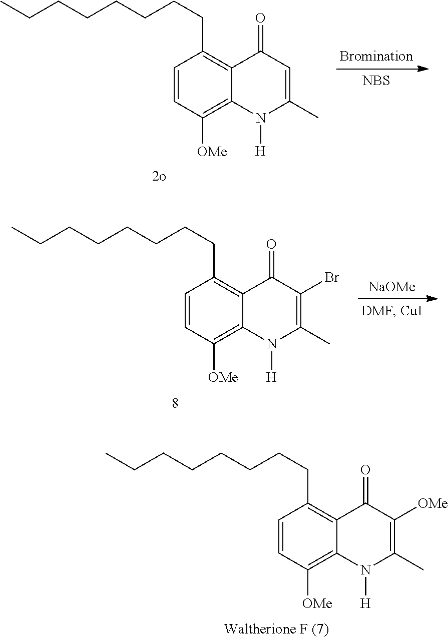

### LIST OF ABBREVIATIONS

HPLC=High pressure Liquid chromatography
TLC=Thin layer chromatography
NMR=Nuclear Magnetic resonance
UV=Ultra-Violet
HRMS=High resolution mass spectroscopy
GC=Gas chromatography
IR=Infra-red
DCM=Dichloromethane
THF: tetrahydrofuran
DCM: dichloromethane

### Material and Method Used in Experiments

The reagents and chemicals used in this process are bought from AVRA or Spectrochem or Sigma-Aldrich and were used as such without any further purification. In this process, the work-up and purification procedures were carried out with reagent grade solvents. All the reactions/experiments steps were monitored by thin layer chromatography and the crude products obtained were subjected to purification using crystallization or chromatography or distillation or extraction or filtration to get the pure compounds in good yields. Further, all the resultant compounds/products were systematically characterized using various analytical and spectral methods.

### Measurement Method

High-resolution mass spectra (HRMS) were obtained from a Xero-G2-XS-QTOF HRMS instrument and Thermo Fisher Scientific Exactive (APCI) Instrument. Nuclear magnetic resonance (NMR) spectra were recorded on a Bruker 600 or 500 or 400 or 300 MHz in CDCl₃ or DMSO-d₆ solvent. Chemical shifts for ¹H NMR are expressed in parts per million (ppm) relative to tetramethylsilane (δ 0.00 ppm). Chemical shifts for ¹³C NMR are expressed in ppm relative to CDCl₃ (δ 77.0 ppm). Data are reported as follows: chemical shift, multiplicity (s=singlet, d=doublet, dd=doublet of doublets, t=triplet, q=quartet, quin=quintet, sext=sextet, m=multiplet), coupling constant (Hz), and integration.

### EXAMPLES

Following examples are given by way of illustration and therefore should not be construed to limit the scope of the invention.
Example 1: 2-Phenylquinolin-4(1H)-one (2a):
Example 2: 2-(4-Fluorophenyl)quinolin-4(1H)-one (2b):
Example 3: 2-(4-Ethylphenyl)quinolin-4(1H)-one (2c):
Example 4: 2-(4-Methoxyphenyl)quinolin-4(1H)-one (2d):
Example 5: 2-Phenyl-1,8-naphthyridin-4(1H)-one (2e):
Example 6: 2-(2-Fluorophenyl)quinolin-4(1H)-one (2f):
Example 7: 5-Bromo-2-phenylquinolin-4(1H)-one (2g):
Example 8: 6-Phenyl-[1,3]dioxolo[4,5-g]quinolin-8(5H)-one (2h):
Example 9: 6-(2-Fluorophenyl)-[1,3]dioxolo[4,5-g]quinolin-8(5H)-one (2i):
Example 10: 2-(4-Methoxyphenyl)-1,8-naphthyridin-4(1H)-one (2j):
Example 11: 2-(Benzo[d][1,3]dioxol-5-yl)-1,8-naphthyridin-4(1H)-one (2k):
Example 12: 2-Cyclohexylquinolin-4(1H)-one (2l):
Example 13: 2-Methylquinolin-4(1H)-one (2m):
Example 14: 2-Cyclopropylquinolin-4(1H)-one (2n):
Example 15: 8-methoxy-2-methyl-5-octylquinolin-4(1H)-one (2o):
Example 16: 2-(benzo[d][1,3]dioxol-5-yl)-1-methylquinolin-4(1H)-one, graveoline (1):
Example 17: 2-(benzo[d][1,3]dioxol-5-yl)-4-methoxyquinoline, graveolinine (2):
Example 18: 2-Butylquinolin-4(1H)-one, pseudane IV (3):
Example 19: 2-Heptylquinolin-4(1H)-one, pseudane VII (4):
Example 20: 2-Octylquinolin-4(1H)-one, pseudane VIII (5):
Example 21: 2-Dodecylquinolin-4(1H)-one, pseudane XII (6):

### Procedures for the Preparation of Compound of Formula I (2a-2o) as Shown in Scheme 1

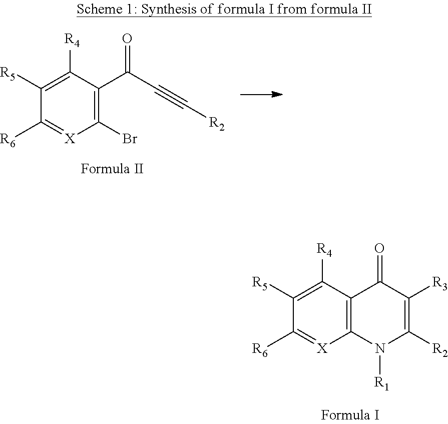

wherein;
X is C, N, and C—OMe,
R₁ is H, and CH₃,
R₂ is C₁-C₁₂ alkyl, cyclopropyl, cyclohexyl, phenyl, 2-fluoro phenyl, 4-fluoro phenyl, 4-methoxy phenyl, 4-ethyl phenyl, and 3,4-methylenedioxyphenyl,
R₃ is H, Br, and OMe,
R₄ is H, C₁-C₈ alkyl, and bromo,
R₅ and R₆ is H, and
R₅-R₆ is —OCH₂O—.

General procedure 1: To a stirred solution of ynone of formula H (0.2 mmol) in aprotic polar solvent such as formamide, N,N,dimethyl formamide (1.5 mL) in Ace pressure tube (Sigma) at room temperature were added ammonia source such as ammonia or ammonium carbonate (1.0 mmol) and metal halide such as copper iodide (0.02 mmol), the cap was closed tightly and the reaction mixture was heated at 100° C. in a preheated oil bath for 12 h. After which the reaction mixture was allowed to cool to room temperature, diluted with EtOAc (5 mL) and cold water (5 mL), layers were separated and the aqueous layer was extracted with EtOAc (5 mL×2). The combined organic extract was washed with brine solution (5 mL) and dried over Na₂SO₄, volatiles were removed under reduced pressure and the obtained crude compound was purified by silica gel column chromatography to afford quinolones (2a-2q) and (3,4,5,6).

### Example 1: 2-Phenylquinolin-4(1H)-one (2a)

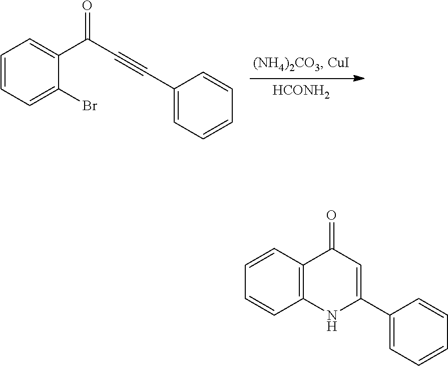

To a stirred solution of ynone 1a (57.0 mg, 0.2 mmol) in formamide (1.5 mL) in Ace pressure tube (Sigma) at room temperature were added ammonium carbonate (97 mg, 1.0 mmol) and copper iodide (4.0 mg, 0.02 mmol), the cap was closed tightly and the reaction mixture was heated at 100° C. in a preheated oil bath for 12 h. After which the reaction mixture was allowed to cool to room temperature, diluted with EtOAc (5 mL) and cold water (5 mL), layers were separated and the aqueous layer was extracted with EtOAc (5 mL×2). The combined organic extract was washed with brine solution (5 mL) and dried over Na₂SO₄, volatiles were removed under reduced pressure and the obtained crude compound was purified by silica gel column chromatography to afford quinolone 2a was prepared as a pale brown solid (35.4 mg, 80%); R_{f}=0.3 (50% EtOAc+Hexane); ¹H NMR (400 MHz, DMSO-d6) δ 11.72 (s, 1H), 8.11 (dd, J=8.1, 1.3 Hz, 1H), 7.87-7.81 (m, 2H), 7.78 (d, J=8.3 Hz, 1H), 7.68 (ddd, J 8.4, 7.0, 1.5 Hz, 1H), 7.63-7.56 (i, 3H), 7.35 (t, J 7.4 Hz, 1H), 6.34 (s, 1H); ¹³C NMR (101 MHz, DMSO-d6) δ 177.43, 150.48, 141.01, 134.71, 132.28, 130.93, 129.49, 127.90, 125.36, 125.21, 123.74, 119.21, 107.83. IR (neat): νₘₐₓ 3545, 2922, 1692, 1627, 1589, 1502, 756 [symbol] ; HRMS (ESIMS): calcd. for C₁₅H₁₂NO [M+H]⁺: calcd m/z 222.0919; found: 222.0912.

The compounds of formula 2b-2o were synthesized following the procedure described above under example 1(2a) and general procedure involving corresponding reactants of formula II, copper iodide and formamide as solvent.

| | Product/ | | Liquid/solid, M.P. | Yield |
|---|---|---|---|---|
| Example | Compound | Ynone | (° C.) | (%) |
| Example 1 | 2a | 1a | Solid, 255-257 | 80 |
| Example 2 | 2b | 1b | Solid, 254-256 | 76 |
| Example 3 | 2c | 1c | Solid, 256-258 | 81 |
| Example 4 | 2d | 1d | Solid, 280-282 | 79 |
| Example 5 | 2e | 1e | Solid, 222-224 | 74 |
| Example 6 | 2f | 1f | Solid, 238-240 | 78 |
| Example 7 | 2g | 1g | Solid, 250-252 | 77 |
| Example 8 | 2h | 1h | Solid, 320-322 | 68 |
| Example 9 | 2i | 1i | Solid, 254-256 | 73 |
| Example 10 | 2j | 1j | Solid, 220-222 | 75 |
| Example 11 | 2k | 1r | Solid, 318-320 | 71 |
| Example 12 | 2l | 1k | Solid, 244-246 | 80 |
| Example 13 | 2m | 1l | Solid, 210-212 | 71 |
| Example 14 | 2n | 1m | Solid, 160-162 | 75 |
| Example 15 | 20 | 1s | Solid, 118-120 | 78 |
| Example 16 | 1 | 2k | Solid, 188-190 | 71 |
| Example 17 | 2 | 2k | Solid, 115-117 | 68 |
| Example 18 | 3 | 1n | Solid, 148-150 | 79 |
| Example 19 | 4 | 1o | Solid, 135-137 | 82 |
| Example 20 | 5 | 1p | Solid, 128-130 | 76 |
| Example 21 | 6 | 1q | Solid, 130-132 | 78 |

### Example 16: 2-(benzo[d][1,3]dioxol-5-yl)-1-methylquinolin-4(1H)-one: Graveoline (1)

To a stirring solution of 2k (30 mg, 0.11 mmol) in anhydrous THF (2 mL) at 0° C. were added NaH (9 mg, 0.22) and Mel (18 □L, 0.33) and continued stirring at rt for 3h, quenched with sat. aq. NH₄Cl, diluted with 2 mL of H₂O and extracted with EtOAc (5 mL×3). The combined organic extract was dried over Na₂SO₄, volatiles were removed under reduced pressure to give crude compound which was purified by column chromatography to afford 1 as a pale brown solid (22.4 mg, 71%); R_{f}=0.35 (50% EtOAc+Hexane); Mp: 188-190° C.; ¹H NMR (500 MHz, CDCl₃) δ 8.48 (dd, J=8.0, 1.5 Hz, 1H), 7.70 (ddd, J=8.6, 7.1, 1.6 Hz, 1H), 7.54 (d, J=8.6 Hz, 1H), 7.42 (t, J=7.5 Hz, 1H), 6.90 (dt, J=8.0, 4.7 Hz, 2H), 6.86 (d, J=1.5 Hz, 1H), 6.28 (s, 1H), 6.06 (s, 2H), 3.63 (s, 3H); ¹³C NMR (126 MHz, CDCl₃) δ 162.80, 158.17, 149.10, 148.77, 148.30, 134.86, 130.00, 129.06, 125.24, 121.69, 121.63, 120.30, 108.42, 108.06, 101.40, 97.59, 55.66; IR (neat): νₘₐₓ 2854, 2100, 1622, 1541, 1434, 1201, 1108, 1023, 784; HRMS (ESIMS): calcd. for C₁₇H₁₄NO₃ [M+H]⁺: calcd m/z 280.0974; found: 280.0980.

### Example 17: 2-(benzo[d][1,3]dioxol-5-yl)-4-methoxyquinoline, graveolinine (2)

To a stirring solution of 2k (30 mg, 0.11 mmol) in anhydrous DMF (2 mL) was added K₂CO₃ (30 mg, 0.22 mmol) and Mel (18 □L, 0.33 mmol) at rt, continued stirring at 80° C. for 30 min, quenched with sat. aq. NH₄Cl, diluted with 2 mL of H₂O and extracted with EtOAc (5 mL×3). The combined organic extract was dried over Na₂SO₄, volatiles were removed under reduced pressure to give crude compound which was purified by column chromatography to afford 2 as a pale brown solid (21.5 mg, 68%); R_{f}=0.5 (50% EtOAc+Hexane); Mp: 115-117° C.; ¹H NMR (400 MHz, DMSO) δ 11.54 (s, 1H), 8.08 (dd, J=8.0, 1.4 Hz, 1H), 7.75 (d, J=8.1 Hz, 1H), 7.66 (ddd, J=8.4, 6.9, 1.5 Hz, 1H), 7.43 (d, J=1.8 Hz, 1H), 7.38 (dd, J=8.1, 1.9 Hz, 1H), 7.35-7.29 (m, 1H), 7.13 (d, J=8.1 Hz, 1H), 6.30 (d, J=1.8 Hz, 1H), 6.16 (s, 2H), 3.32 (s, 3H); ¹³C NMR (101 MHz, CDCl₃) δ 162.81, 158.15, 149.06, 148.77, 148.30, 134.80, 130.01, 129.02, 125.24, 121.70, 121.62, 120.30, 108.42, 108.05, 101.39, 97.58, 55.66; IR (neat): νₘₐₓ 2776, 1728, 1597, 1498, 1409, 1239, 1045, 815; HRMS (ESIMS): calcd. for C₁₇H₁₄NO₃[M+H]⁺: calcd m/z 280.0974; found: 280.0975.

### Example 18: 2-Butylquinolin-4(1H)-one, Pseudane IV (3)

By following general procedure 1, with ynone 1n, pseudane IV (3) was prepared as a pale brown solid (31.7 mg, 79%).

### Example 19: 2-Heptylquinolin-4(1H)-one, Pseudane VII (4)

By following general procedure 1, with ynone 1o, pseudane VII (4) was prepared as a pale brown solid (39.8 mg, 82%); R_{f}=0.4 (50% EtOAc+Hexane).

### Example 20: 2-Octylquinolin-4(1H)-one, Pseudane VIII (5)

By following general procedure 1, with ynone 1p, pseudane VIII (5) was prepared as a pale brown solid (39.0 mg, 76%); R_{f}=0.4 (50% EtOAc+Hexane).

### Example 21: 2-Dodecylquinolin-4(1H)-one, Pseudane XII (6)

By following general procedure 1, with ynone 1q, pseudane XII (6) was prepared as a pale brown solid (48.8 mg, 78%); R_{f}=0.4 (50% EtOAc+Hexane).

The invention also provides a concise approach for the total synthesis of waltherione F (7) from one of the product (2o) obtained above and is described as follows:

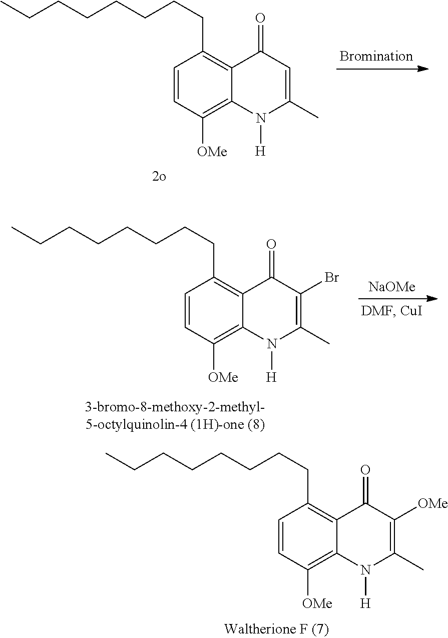

To a stirring solution of 8-Methoxy-2-methyl-5-octylquinolin-4(1H)-one (2o) (100 mg, 0.31 mmol) in acetonitrile (8 mL) was added N-bromosuccinimide (60 mg, 0.35 mmol) in acetonitrile (5 mL) and were further stirred at room temperature. After stirring for 2 hours, the reaction mixture was diluted with CH₂Cl₂ (20 mL), washed with water (2×10 mL), aq. layer was extracted with dichloromethane (10 mL), dried over Na₂SO₄ and concentrated under reduced pressure to afford 3-bromo-8-methoxy-2-methyl-5-octylquinolin-4 (1H)-one as a pale brown solid, (79 mg, 70%). 3-bromo-8-methoxy-2-methyl-5-octylquinolin-4 (1H)-one (79 g, 0.22 mmol) in DMF (5 mL), NaOMe (0.20 mL, 1.08 mmol) and CuI (20 mg, 0.11 mmol) were put in a 50 ml round-bottom flask. The mixture was heated to 120° C. and then was left to stir for 2 hours. After completion of the reaction, the reaction mixture was filtered through celite and the filtrate was concentrated under reduced pressure which afforded a yellow solid. The product was purified through column chromatography on silica gel (0-7% methanol in dichloromethane) to afford waltherione F (7) pale yellow solid, (47 mg, 62%). M.p. 110-112° C.; ¹H NMR (500 MHz, MeOD₄) δ 7.02 (d, J=8.1 Hz, 1H), 6.93 (d, J=8.1 Hz, 1H), 4.00 (s, 3H), 3.77 (s, 3H), 3.28-3.23 (m, 2H), 2.48 (s, 3H), 1.59 (dt, J=15.2, 7.4 Hz, 2H), 1.42-1.35 (m, 2H), 1.34-1.22 (m, 10H), 0.88 (t, J=7.0 Hz, 3H); ¹³C NMR (101 MHz, MeOD₄) δ 175.93, 147.86, 143.21, 142.79, 136.73, 132.40, 125.38, 125.02, 110.57, 60.23, 56.54, 36.35, 33.72, 33.08, 30.90, 30.80, 30.56, 23.75, 14.44, 14.11; IR (neat): νₘₐₓ 2920, 1715, 1621, 1570, 1521, 1241, 1182, 1021, 810, 668; HRMS (ESIMS): calcd. for C₂₀H₃₀NO₃[M+H]⁺: calcd m/z 332.226; found: 332.226.

### Preparation of 2-bromoaryl-ynones of Formula II

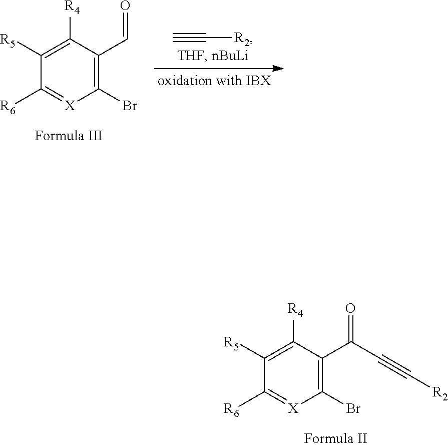

Wherein X═C, N, C—OMe, R₁═H, CH₃, R₂═C₁-C₁₂ alkyl, cyclopropyl, cyclohexyl, phenyl, 2-fluoro phenyl, 4-fluoro phenyl, 4-methoxy phenyl, 4-ethyl phenyl, 3,4-methylenedioxyphenyl. R₄═H, C₁-C₈ alkyl, Bromo, R₅═H, R₆═H, R₅-R₆═—OCH₂O—.

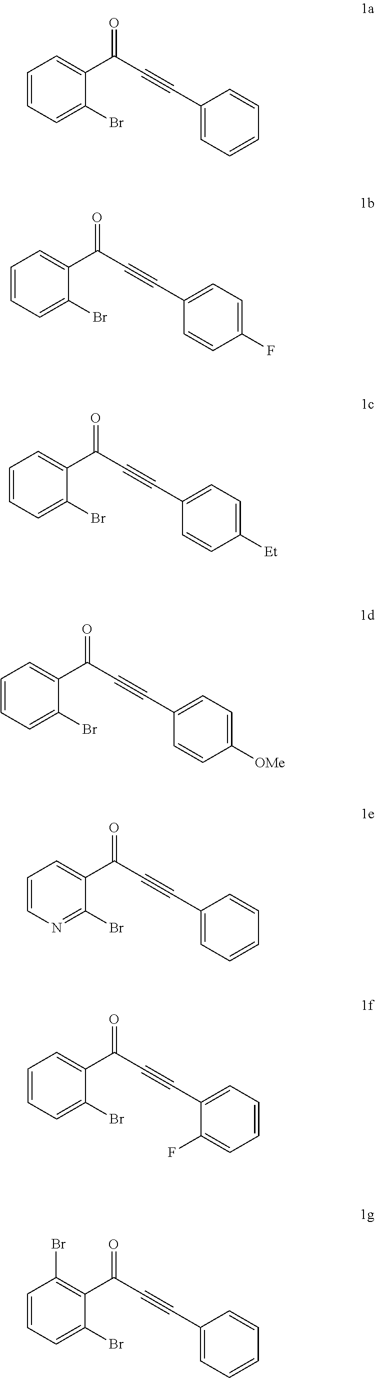

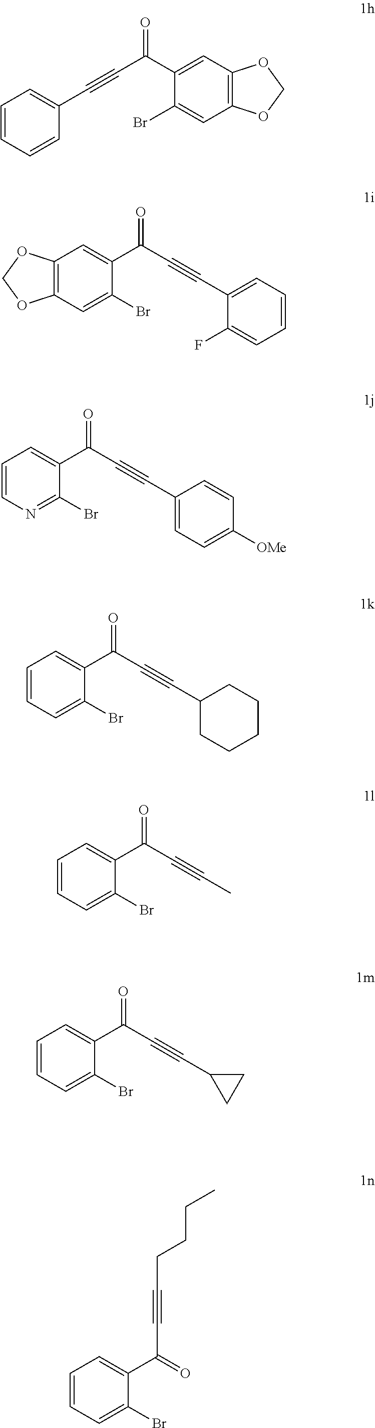

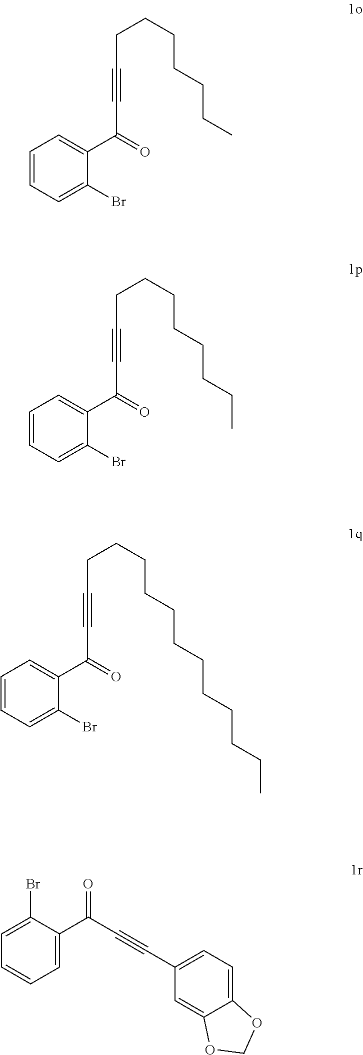 [SMILES: CCCCCCCC#CC(=O)c1ccccc1Br ; CCCCCCCCC#CC(=O)c1ccccc1Br ; CCCCCCCCCCCCC#CC(=O)c1ccccc1Br ; O=C(C#Cc1ccc2c(c1)OCO2)c1ccccc1Br]

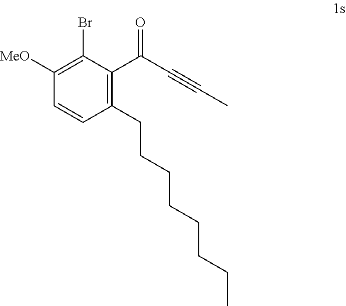 [SMILES: CC#CC(=O)c1c(CCCCCCCC)ccc(OC)c1Br]

General procedure 2: For the preparation of 2-bromoarylynones (1a-1s) of formula II utilized for the synthesis of representative quinolones:

Base like LiHMDS, or n-BuLi (1.6 M, 5 mmol) was added to a stirring solution of alkyne (6 mmol) in anhydrous THF (30 mL) at −25° C. to −15° C., and the resulting reaction mixture was stirred for another 15 min-half an hour. at the same temperature. To this 2-bromo aryl aldehyde (5 mmol) in THF (5 mL) was added drop wise and allowed to warm to room temperature and the reaction was monitored by TLC. After complete consumption of the starting material (monitored by TLC), the reaction mixture was quenched by drop wise addition of saturated aq. NH4Cl (10 mL) solution and diluted with H₂O (40 mL) and EtOAc (20 mL). The layers were separated and the aqueous layer was extracted with EtOAc (3×10 mL). The combined organic layer was washed with brine solution and dried over anhydrous Na₂SO₄, concentrated under reduced pressure to afford the crude product, which was purified by column chromatography (EtOAc/hexane, 1:5) to furnish the propargyl alcohol. To a stirred solution of propargyl alcohol (10 mmol) in DMSO (20 mL) at room temperature was added IBX (12 mmol) and the reaction mixture was stirred for 2-3 h. After complete consumption of the starting material (monitored by TLC), the reaction mixture was filtered through celite with the aid of EtOAc and the resulting filtrate was washed with cold H₂O (25 mL×2), EtOAc (30 mL) and brine solution (10 mL) and the organic layer was dried over anhydrous Na₂SO. Volatiles were removed under reduced pressure and the obtained crude mixture was purified by silica gel column chromatography (EtOAc/hexane 1:5) to yield the brominated substituted ynones.

By following the general procedure 2 and corresponding specific starting materials, following compounds were prepared.

| | Product/ | Physical state, M.P. | Yield |
|---|---|---|---|
| Example | Compound | (° C.) | (%) |
| Example 22 | 1a | Liquid | 72 |
| Example 23 | 1b | Solid, 73-75 | 68 |
| Example 24 | 1c | Liquid | 70 |
| Example 25 | 1d | Liquid | 65 |
| Example 26 | 1e | Solid, 72-74 | 67 |
| Example 27 | 1f | Liquid | 63 |
| Example 28 | 1g | Solid, 78-80 | 70 |
| Example 29 | 1h | Solid, 98-100 | 68 |
| Example 30 | 1i | Solid, 125-127 | 62 |
| Example 31 | 1j | Solid, 116-118 | 69 |
| Example 32 | 1k | Liquid | 72 |
| Example 33 | 1l | Liquid | 78 |
| Example 34 | 1m | Liquid | 70 |
| Example 35 | 1n | Liquid | 75 |
| Example 36 | 1o | Liquid | 71 |
| Example 37 | 1p | Liquid | 74 |
| Example 38 | 1q | Liquid | 70 |
| Example 39 | 1r | Solid, 112-114 | 69 |
| Example 40 | 1s | Liquid | 80 |

Example 33: 1-(2-bromophenyl)but-2-yn-1-one (11): To a stirring solution of 2-bromobenzaldehyde (925 mg, 5.0 mmol) in 10 mL of THF at 0° C. was added was added 1-propynylmagnesium bromide (12.0 mL, 0.5 M in THF, 6.0 mmol) and stirred for 1 h, quenched with sat. aq. NH₄Cl solution (5 mL) and diluted with water (25 mL) and the organic layer was extracted using EtOAc (2×25 mL), the combined organic extract was dried over Na₂SO₄ and concentrated under reduced pressure to give the crude secondary alcohol, used for next reaction without further purification. To a stirred solution of propargyl alcohol (5.0 mmol) in DMSO (15 mL) at room temperature was added IBX (1.68 g, 6.0 mmol) and the reaction mixture was stirred for 2 h. The reaction mixture was filtered through celite with the aid of EtOAc (25 mL) and the resulting filtrate was washed with cold H₂O (25 mL×2), the aq. layer was extracted with EtOAc (25 mL) and the combined organic extract was washed with brine solution (25 mL) and the dried over anhydrous Na₂SO₄. Volatiles were removed under reduced pressure and the obtained crude mixture was purified by silica gel column chromatography (EtOAc/hexane 1:5) to yield the bromo-ynones (11) as colourless liquid (870 mg, 78%).

### Example 40: 1-(2-Bromo-3-methoxy-6-octylphenyl)but-2-yn-1-one (1s)

A two neck round bottomed flask was degassed under high vacuum, diisopropylamine (0.64 mL, 4.52 mmol) was added in anhydrous THF (10.0 mL) under N₂, n-BuLi (2.8 mL, 1.6 M in THF, 4.52 mmol) was added dropwise to the stirred solution at −78° C. After stirring 30 min, 2,4-dibromo-1-methoxybenzene (1.0 g, 3.77 mmol) in 10 mL THF and DMF (0.21 mL, 2.65 mmol) were added subsequently to the yellow suspension, the reaction mixture was further stirred at room temperature for 15 minutes after which it was quenched with saturated solution of ammonium chloride (10 mL). The reaction mixture was diluted with water (25 mL) and the organic layer was extracted using EtOAc (2×25 mL), the combined organic extract was dried over Na₂SO₄ and concentrated under reduced pressure to give the crude compound which was purified through column chromatography on silica gel to afford dibromo-aldehyde as pale yellow solid (800 mg, 73%); Mp: 120-122° C.; ¹H NMR (500 MHz, CDCl₃) δ 10.29 (s, 1H), 7.73 (d, J=9.0 Hz, 1H), 6.88 (d, J=9.0 Hz, 1H), 3.91 (s, 4H); ¹³C NMR (126 MHz, CDCl₃) δ 190.20, 160.13, 137.69, 126.52, 126.29, 118.14, 112.49, 56.50; IR (neat): νₘₐₓ 2886, 1692, 1574, 1456, 1380, 1267, 1184, 1128, 1033, 814, 767; HRMS (ESIMS): calcd. for C₈H₇O₂Br₂ [M+H]⁺: calcd m/z 292.8813; found: 292.8828; An ace pressure tube was charged with 2,6-dibromo-3-methoxybenzaldehyde (750 mg, 2.56 mmol), n-octylboronic acid (485 mg, 3.07 mmol) after degassing the tube, Pd(PPH₃)₄ (148 mg, 0.13 mmol), and K₂CO₃ (530 mg, 3.84 mmol) were added successively and anhydrous toluene (12 mL). The tube was sealed carefully and the reaction mixture was heated on oil bath at 100° C. for a period of 12 h. After cooling to room temperature, the reaction mixture was filtered through a celite and the filtrate was concentrated under reduced pressure to yield the crude compound which was subjected to purification through column chromatography to yield coupled product as colorless liquid (544 mg, 65%). ¹H NMR (500 MHz, CDCl₃) δ 10.52 (s, 1H), 7.65 (d, J=8.9 Hz, 1H), 6.73 (d, J=8.9 Hz, 1H), 3.88 (s, 3H), 3.05 (dd, J=9.2, 6.6 Hz, 2H), 1.54-1.20 (m, 12H), 0.88 (t, J=7.0 Hz, 3H); ¹³C NMR (126 MHz, CDCl₃) δ 191.56, 162.13, 145.14, 138.20, 124.80, 117.75, 110.64, 56.03, 32.76, 31.94, 29.96, 29.86, 29.35, 29.33, 22.73, 14.16; IR (neat): νₘₐₓ 2927, 2857, 1690, 1575, 1460, 1408, 1270, 1173, 1100, 811, 768; HRMS: (ESIMS): calcd. for C₁₆H₂₄O₂Br[M+H]⁺: calcd m/z 327.0960; found: 327.0969.

To a stirring solution of octyl-bromo-aldehyde (530 mg, 1.62 mmol) in 10 mL of THF at 0° C. was added was added 1-propynylmagnesium bromide (3.9 mL, 0.5 M in THF, 1.95 mmol) and stirred for 1 h, quenched with sat. aq. NH₄Cl solution (5 mL) and diluted with water (25 mL) and the organic layer was extracted using EtOAc (2×25 mL), the combined organic extract was dried over Na₂SO₄ and concentrated under reduced pressure to give the crude secondary alcohol, which was used for next reaction without further purification. To a stirred solution of propargyl alcohol (595 mg, 1.62 mmol) in DMSO (10 mL) at room temperature was added IBX (545 mg, 1.95 mmol) and the reaction mixture was stirred for 2 h. The reaction mixture was filtered through celite with the aid of EtOAc (25 mL) and the resulting filtrate was washed with cold H₂O (25 mL×2), the aq. layer was extracted with EtOAc (25 mL) and the combined organic extract was washed with brine solution (25 mL) and the dried over anhydrous Na₂SO₄. Volatiles were removed under reduced pressure and the obtained crude mixture was purified by silica gel column chromatography (EtOAc/hexane 1:5) to yield the bromo-ynone (Is) as colourless liquid (473 mg, 80%). ¹H NMR (500 MHz, CDCl₃) δ 7.49 (d, J=8.8 Hz, 1H), 6.66 (d, J=8.8 Hz, 1H), 3.81 (s, 3H), 2.71-2.61 (m, 2H), 2.05 (s, 3H), 1.60-1.51 (m, 1H), 1.40-1.21 (m, 11H), 0.88 (t, J=7.0 Hz, 3H); ¹³C NMR (126 MHz, CDCl₃) δ 181.10, 155.84, 139.90, 134.34, 131.57, 116.04, 110.59, 92.80, 81.71, 56.13, 33.34, 31.89, 29.85, 29.26, 22.71, 14.15, 4.54; IR (neat): νₘₐₓ 2926, 2857, 2225, 1659, 1575, 1461, 1274, 1091, 923, 809, 765; HRMS (ESIMS): calcd. for C₁₉H₂₆O₂Br[M+H]⁺: calcd m/z 365.116; found: 365.1135.

### Significance of the Work Carried Out

In view of the importance of quinolones, a new and efficient process for the preparation of quinolones from 2-bromoaryl-ynones and ammonium carbonate as ammonia source in presence of CuI is presented. The ynones can be easily accessible from the readily available commercial materials in two step process. The present process method synthesis of substituted quinolones of formula I by us serves as a highly effective new method for the synthesis of several quinolones and process for the synthesis of natural products pseudane IV, VII, VIII, XII and waltherione F respectively thereof.

### Advantages of the Invention

The various advantages of the present process are given below.
1. The present process serves as a highly efficient and scalable production method for the preparation of quinolones and analogs, in particular quinolones and analogs by amine insertion method.
2. The advantage of the present invention is that the process could be operated by one-pot employing ammonia source and an additive.
3. Another advantage of the present invention is, it includes very highly feasible reaction parameters.
4. Isolation and/or purification of the product/s is straight forward.
5. This is an attractive and economic method for the production of quinolones and analogs, in particular quinolones.
6. This process could be adopted to generate a large library of process intermediates and quinolones analogues.
7. The process directly leads to the synthesis of graveoline
8. The process directly leads to the synthesis of graveolinine
9. The process directly leads to the synthesis of pseudane IV
10. The process directly leads to the synthesis of pseudane VII
11. The process directly leads to the synthesis of pseudane VIII
12. The process directly leads to the synthesis of pseduane XII
13. Another advantage of this process involves utility of quionolone synthesized by the developed process for the natural product waltherione F synthesis

We claim:

1. A process for the preparation of compound of Formula (I), the process comprising the steps of: 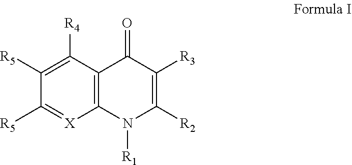
employing an additive and ammonia source in one-pot approach to pre-installed ynones of Formula (II) 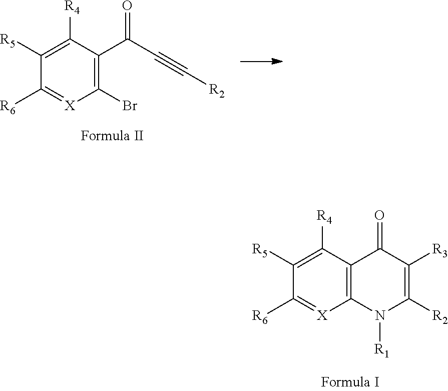
wherein;
X is C, N, and C—OMe,
R₁ is H, and CH₃,
R₂ is C₁-C₁₂ alkyl, cyclopropyl, cyclohexyl, phenyl, 2-fluoro phenyl, 4-fluoro phenyl, 4-methoxy phenyl, 4-ethyl phenyl, and 3,4-methylenedioxyphenyl,
R₃ is H, Br, and OMe,
R₄ is H, C₁-C₈ alkyl, and bromo,
R₅ and R₆ is H, and
R₅ and R₆ can be taken together to form —OCH₂O—;

2. The process for the preparation of compound of formula (I) as claimed in claim 1, wherein the compound of formula (I) is selected from the group consisting of:

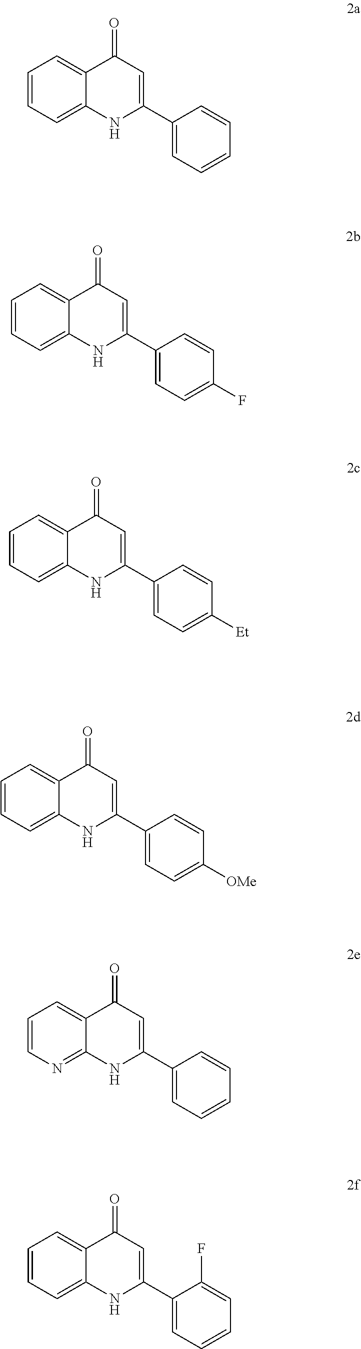 [SMILES: O=c1cc(-c2ccccc2)[nH]c2ccccc12 ; O=c1cc(-c2ccc(F)cc2)[nH]c2ccccc12 ; CCc1ccc(-c2cc(=O)c3ccccc3[nH]2)cc1 ; COc1ccc(-c2cc(=O)c3ccccc3[nH]2)cc1 ; O=c1cc(-c2ccccc2)[nH]c2ncccc12 ; O=c1cc(-c2ccccc2F)[nH]c2ccccc12]

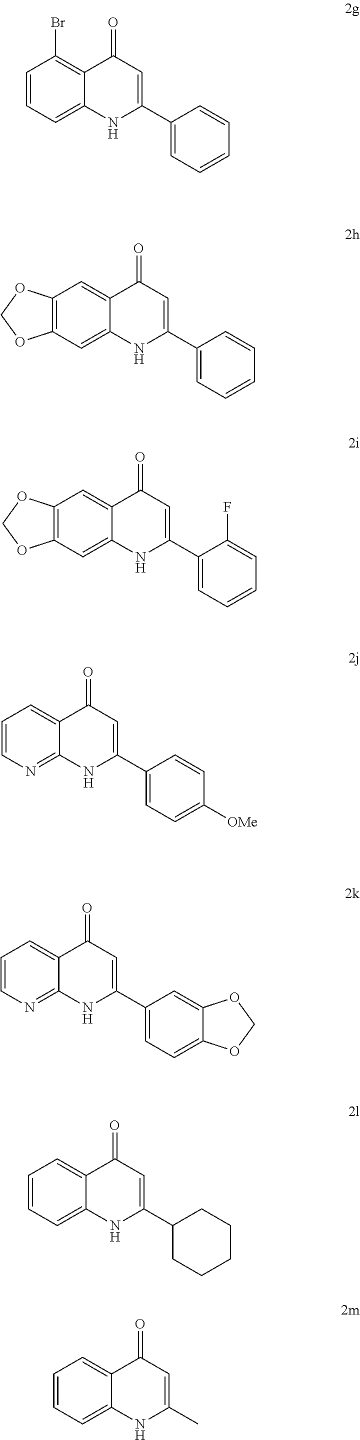

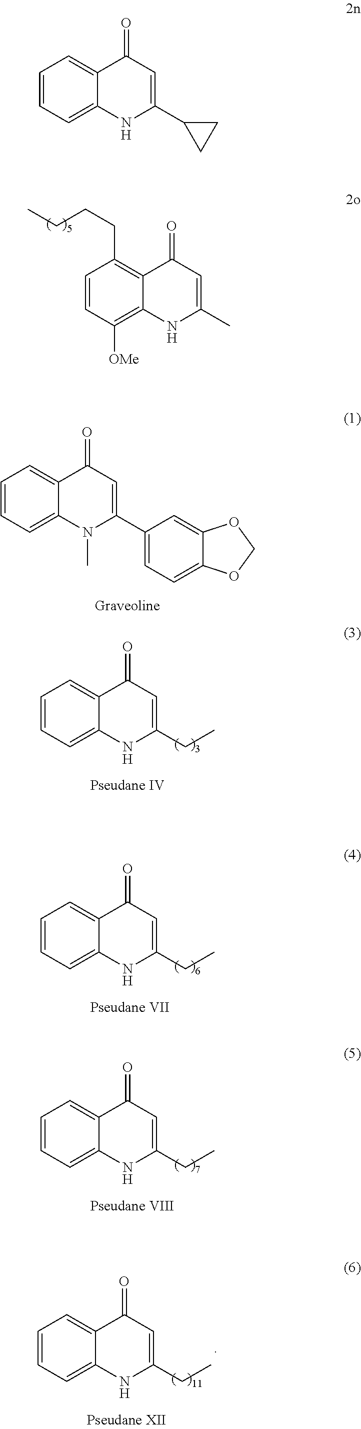

3. A compound of formula (2g)

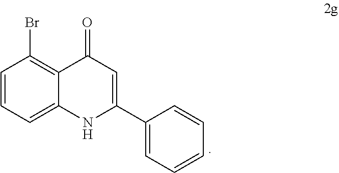 [SMILES: O=c1cc(-c2ccccc2)[nH]c2cccc(Br)c12]

4. A compound Graveolinine (2) derived from compound 2k of formula (I)

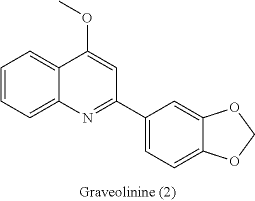 [SMILES: COc1cc(-c2ccc3c(c2)OCO3)nc2ccccc12]

5. The process as claimed in claim 1 comprising, reacting bromoaryl ynones of formula (II) with inorganic base in a polar solvent and heating the mixture along with ammonia source in presence of an additive at 80-120° C. for 8-15 h to obtain quinolones of formula (I).

6. The process as claimed in claim 5, wherein the inorganic base is selected from the group consisting of cesium carbonate, DBU, potassium carbonate, sodium carbonate, potassium bicarbonate, sodium bicarbonate, potassium phosphate, and ammonium carbonate and the polar solvent is selected from the group consisting of ethers, alcohols, esters, dimethylformamide, formamide, dimethylsulfoxide, and acetonitrile.

7. The process as claimed in claim 5, wherein the ammonia source is selected from ammonium chloride, ammonium acetate, ammonium carbonate and ammonium formate and the additive is selected from copper iodide, copper bromide, copper chloride, and copper acetate.

8. A compound of general formula (II), 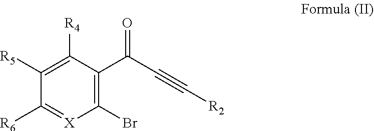
wherein;
X is C, N, and C—OMe,
R₂ is C₁-C₁₂ alkyl, cyclopropyl, cyclohexyl, phenyl, 2-fluoro phenyl, 4-fluoro phenyl, 4-methoxy phenyl, 4-ethyl phenyl, and 3,4-methylenedioxyphenyl,
R₄ is H, C₁-C₈ alkyl, and bromo,
R₅ and R₆ is H, and
R₅ and R₆ can be taken together to form —OCH₂O—.

9. The compound of formula (II) as claimed in claim 8 selected from the group consisting of

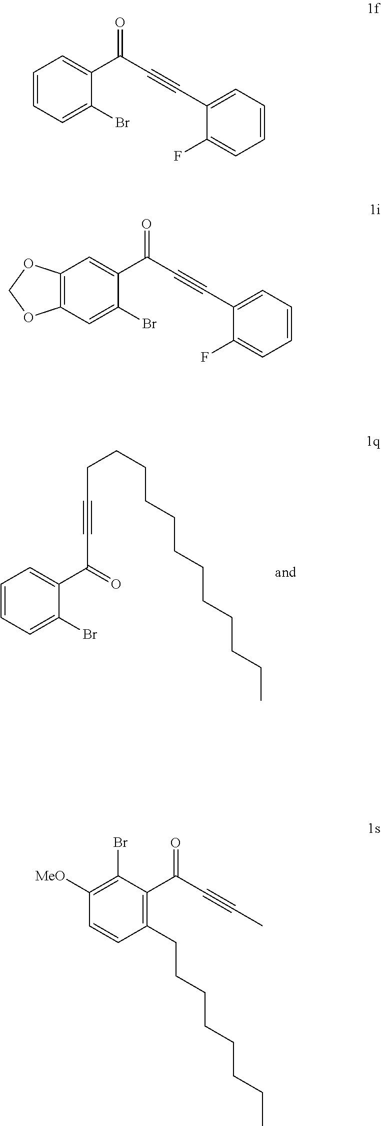 [SMILES: O=C(C#Cc1ccccc1F)c1ccccc1Br ; O=C(C#Cc1ccccc1F)c1cc2c(cc1Br)OCO2 ; CCCCCCCCCCCCC#CC(=O)c1ccccc1Br ; CC#CC(=O)c1c(CCCCCCCC)ccc(OC)c1Br]

10. A process for the preparation of graveoline (1), graveolinine (2), pseudane IV (3), pseudane VII (4), pseudane VIII (5), and pseudane XII (6) of following formulae: 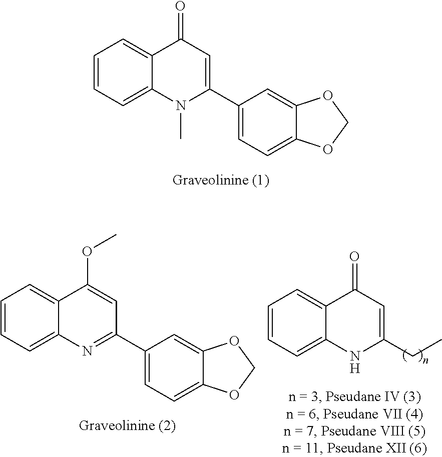
comprising: the treatment of ynones of formula (II) 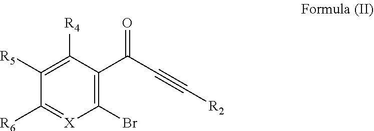
wherein;
X is C, N, and C—OMe,
R₂ is C₁-C₁₂ alkyl, cyclopropyl, cyclohexyl, phenyl, 2-fluoro phenyl, 4-fluoro phenyl, 4-methoxy phenyl, 4-ethyl phenyl, and 3,4-methylenedioxyphenyl,
R₄ is H, C₁-C₈ alkyl, and bromo,
R₅ and R₆ is H, and
R₅ and R₆ can be taken together to form —OCH₂O—
with ammonia source in presence of metal halide as an additive in polar solvent selected from DMF or formamide.

11. A process for the preparation of waltherione F of formula (7) comprising the steps of: 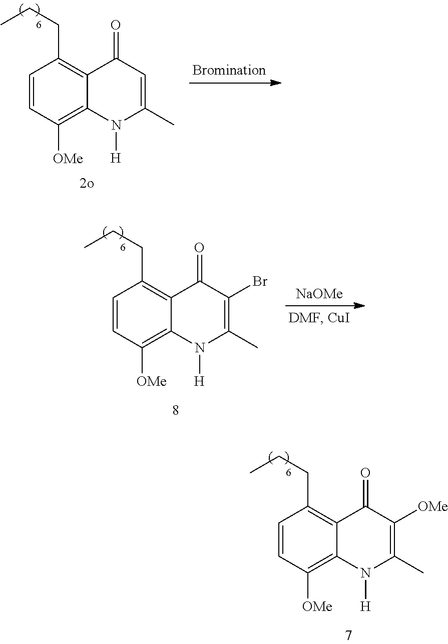
i) subjecting 8-methoxy-2-methyl-5-octylquinolin-4(1H)-one (2o) to bromination to provide the corresponding brominated product (8), and
ii) treating brominated product (8) with sodium methoxide in presence of copper iodide to obtain waltherione F(7).
